(19) United States
(12) Patent Application Publication
(10) Pub. No.: US 2022/0165431 A1
(43) Pub. Date: May 26, 2022

(54) SLEEP DISORDER DATA FIDELITY MANAGEMENT SYSTEM

(71) Applicant: KONINKLIJKE PHILIPS N.V., EINDHOVEN (NL)

(72) Inventors: Gary Nelson GARCIA MOLINA, Cambridge, MA (US); Yash Parag MOKASHI, Pittsburgh, PA (US); Jesse SALAZAR, Gibsonia, PA (US); Monica Helen BUSH, Murrysville, PA (US); Kousalya RONDINELLI, Cranberry Township, PA (US); Jenna Marie SCHABDACH, Pittsburgh, PA (US)

(21) Appl. No.: 17/437,077

(22) PCT Filed: Feb. 12, 2020
(86) PCT No.: PCT/AU2020/050118

§ 371 Date: Sep. 8, 2021

Related U.S. Application Data
(60) Provisional application No. 62/815,562, filed on Mar. 8, 2019.

(51) Int. Cl. G16H 50/70; G06F 16/903; G06F 16/908; G06F 16/906

(52) U.S. Cl. CPC G16H 50/70; G06F 16/906; G06F 16/908; G06F 16/90335

(57) ABSTRACT

Some embodiments relate to computer-implemented methods and systems for fidelity improvement of sleep disorder data. An example method comprises: a server system transmitting query program code defining a plurality of queries to a client device, the plurality of queries relating to sleep disorders, wherein: at least two queries of the plurality of queries relate to a common sleep metric, and at least one other of the plurality of queries relates to a different sleep metric, and the query program code is executable by the client device to permit the client device to transmit at least one response object encoding a response to each of the at least two queries; the server system receiving the at least one response object from the client device and determining the response to each of the at least two queries encoded in the response object; determining a difference between the responses to the at least two queries that relate to a common sleep metric; responsive to determining that the difference is greater than a predetermined difference threshold, the server system transmitting further query program code defining a further query that relates to the common sleep metric, the further query program code being executable by the client device to permit or cause the client device to transmit a further response object encoding a further response to the further query, wherein the further query is selected so that a difference between the further response and one of the at least two queries is less than the predetermined difference threshold.

### TECHNICAL FIELD

Embodiments generally relate to data fidelity management and improvement systems and methods for sleep disorder related data.

### BACKGROUND

A number of distinct, clinically validated questionnaires have been developed to support the classification and diagnoses of individual sleep problems in large populations. While these questionnaires are useful in isolation, combining these tools in conjunction with other subjective and objective data points allows for the development of an automated system for self-driven classification of individual, as well as comorbid sleep problems and suggest solutions.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### SUMMARY

Some embodiments relate to a computer-implemented method for fidelity improvement of sleep disorder data, the method comprising: a server system transmitting query program code defining a plurality of queries to a client device, the plurality of queries relating to sleep disorders, wherein: at least two queries of the plurality of queries relate to a common sleep metric, and at least one other of the plurality of queries relates to a different sleep metric; and the query program code is executable by the client device to permit the client device to transmit at least one response object encoding a response to each of the at least two queries; the server system receiving the at least one response object from the client device and determining the response to each of the at least two queries encoded in the response object; determining a difference between the responses to the at least two queries that relate to a common sleep metric; responsive to determining that the difference is greater than a pre-determined difference threshold, the server system transmitting further query program code defining a further query that relates to the common sleep metric, the further query program code being executable by the client device to permit or cause the client device to transmit a further response object encoding a further response to the further query, wherein the further query is selected so that a difference between the further response and one of the at least two queries is less than the predetermined difference threshold; the server system receiving the further response object from the client device and determining the further response encoded in the further response object. In some embodiments, the further query may comprise a query that is substantially identical or similar to one of the at least two queries.

The method may further comprise determining the difference threshold, wherein the difference threshold is determined based on response values of a plurality of stored response objects related to the common sleep metric accessible to the server system.

The common sleep metric and the different sleep metric may be part of a plurality of sleep metrics, and each sleep metric may have a respective difference threshold associated therewith.

The further query program code directed to a sensor and the further response object may comprise health data from the sensor. The client device may comprise the sensor or the sensor may be separate from the client device. If separate, then the client device may communicate with the sensor to receive data content of the further response object. Alternatively, for a sensor that is separate from the client device, the data content of the further response may be received from a server that has access to stored data received from that sensor.

The health data may comprise one or more of: cardiac activity measurement data, physical activity measurement data, blood pressure measurement data, respiratory activity measurement data, brain activity (e.g. electroencephalogram) data, eye movement data, sleep stage data (e.g. polysomnography data), heart rate data, movement data, breathing sound data, breathing rate data or other longitudinal biological measures.

Some embodiments relate to a computer-implemented method for revising queries to improve fidelity of sleep disorder data, the method comprising: retrieving infidelity metric data in relation to a plurality of queries relating to sleep disorders; for each query in the plurality of queries that has an infidelity metric value greater than a predetermined infidelity threshold, generating a word space vector of the query; enumerating all possible word combinations in each of the word space vectors; counting occurrence of each enumerated word combination across all of the word space vectors; identifying a subset of the enumerated word combinations having an occurrence greater than an occurrence threshold; revising each query in the plurality of queries where the identified subset of the enumerated word combinations occur to improve the fidelity of responses to each revised query. Generation of a word space vector may also be referred to as word embedding.

The generated word space vector that may be a sparse vector, for example.

The method may further comprise a clustering module of the server system identifying one or more clusters of sleep disorder user data that most closely relate to the retrieved infidelity metric data; wherein the revising comprises, for each query in the plurality of queries , revising that query for each of the identified one or more clusters.

The infidelity metric data may comprise health data from a sensor. The health data may comprise one or more of: cardiac activity measurement data, physical activity measurement data, blood pressure measurement data, respiratory activity measurement data, brain activity (e.g. electroencephalogram) data, eye movement data, sleep stage data (e.g. polysomnography data), heart rate data, movement data, breathing sound data or breathing rate data.

Some embodiments relate to a system for fidelity improvement of sleep disorder data, the system comprising a server system comprising: one or more processors; and memory accessible to the one or more processors storing executable program instructions to implement a method for fidelity improvement of sleep disorder data described herein.

Some embodiments relate to a system for revising queries to improve fidelity of sleep disorder data, the system comprising a server system comprising: one or more processors; and memory accessible to the one or more processors storing executable program instructions to implement a method for revising queries to improve fidelity of sleep disorder data described herein.

Some embodiments relate to one or more computer-readable media storing computer-executable instructions that, when executed by one or more computers, direct the one or more computers to perform any one of the methods for revising queries to improve fidelity of sleep disorder data or any one of the methods for fidelity improvement of sleep disorder data described herein.

### BRIEF DESCRIPTION OF THE FIGURES

FIG. 1 is a block diagram of a system for sleep disorder data fidelity management;

FIG. 2 is a flowchart of a method for improving the fidelity of sleep disorder data;

FIG. 3 is a flowchart of a method for revising queries to improve fidelity of sleep disorders data;

FIG. 4A is an example matrix of a map of sleep metrics and queries;

FIG. 4B is an example matrix of a map of sleep metrics and queries, illustrating part of the method of improving the fidelity of sleep disorder data of FIG. 2;

FIG. 5A is an example matrix of a map of sleep metrics and queries showing marginal infidelity values for each query;

FIG. 5B is a schematic diagram including the example matrix of FIG. 5A and illustrating part of the method of revising queries to improve fidelity of sleep disorders data of FIG. 3;

FIG. 6A is an example screenshot of a client device application according to some embodiments; and

FIG. 6B is an example screenshot of a client device application according to some embodiments;

FIG. 6C is an example screenshot of a client device application according to some embodiments; and

FIG. 6D is an example screenshot of a client device application according to some embodiments.

### DETAILED DESCRIPTION

The embodiments generally relate to data fidelity management and improvement systems for sleep disorder related data. Some embodiments rely on information from one or more sensor devices, such as fitness or health related sensor devices to improved data fidelity management systems. Embodiments may rely on redundancy determined according to metrics or vector space maps of query objects to optimize the definition of query objects in order to improve the data fidelity of sleep disorder related data. Sleep disorder data with improved fidelity may allow improved or more accurate sleep disorder identification. The sleep disorder identification may be performed according to embodiments disclosed in U.S. Provisional Patent Application Ser. No. 62/812,066, filed 28 Feb. 2019, the contents of which are hereby incorporated by reference.

Objects as described in this specification, including query objects and response objects may be discretely identifiable computing communication constructs capable of transmission and reception over a computer network. Objects may comprise data, and an object when received by a computing device may be capable of interpretation by the computing device to access the data. Objects may comprise program code, and an object when received by a computing device may be capable of execution by the computing device to execute the program code. In some embodiments, objects may include a combination of data and program code. Object may comprise by one or more header elements and one or more payload elements. The payload elements may comprise the data or the program code or both.

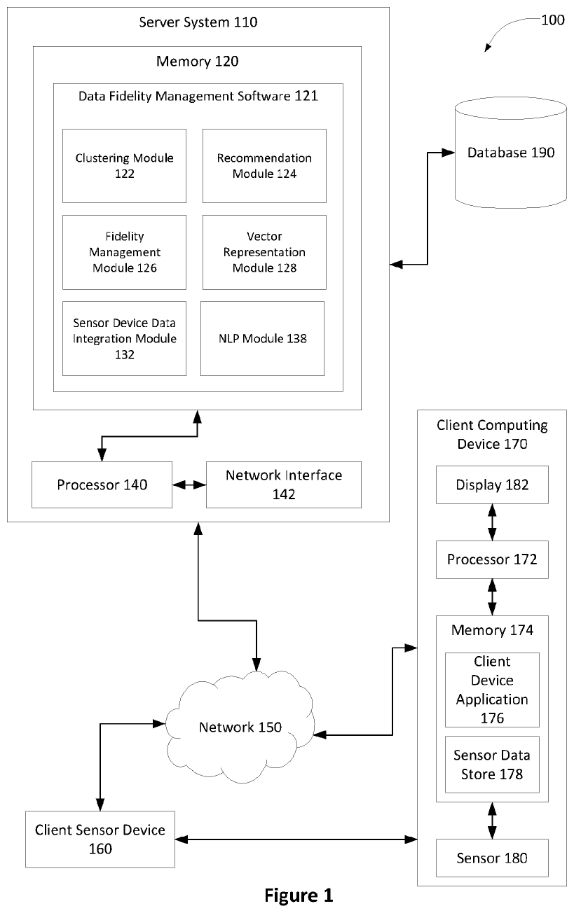

FIG. 1 is a block diagram of a sleep disorder data fidelity management system 100 according to some embodiments. Data fidelity management system 100 comprises a server system 110 that implements a server side part of data fidelity management software 121. A client computing device 170 implements the client side part of the data fidelity management software 121. More than one client computing device 170 may interact with the server system 110 to form a part of the data fidelity management system 100. In some embodiments, one or more client sensor devices 160 may also form part of the decision support system 100. In some embodiments, the client computing device 170 may comprise a sensor data store 178. The sensor data store 178 may store data generated by the one or more client sensor devices 160 and/or a sensor 180 that is part of the client computing device 170. The sensor 180 may be or comprise an audio sensor or a health data tracking sensor that gathers information regarding health data of the end user, for example. The client sensor device 160 may be or comprise a health or sleep related sensor or a fitness tracker, for example. The client sensor device 160 may be a smartwatch, for example, that tracks an individual's health data over time.

The health data may comprise one or more of: cardiac activity measurement data, physical activity measurement data, blood pressure measurement data or respiratory activity measurement data, brain activity (e.g. electroencephalogram) data, eye movement data, sleep stage data (e.g. polysomnography data), or other longitudinal biological measures (i.e. gathered over a period of time of one or more days, weeks or months), for example. Cardiac activity measurement data may include a heart rate measurement, a measure of electrical activity of the heart, or a measure of volumetric changes of the heart, for example. Physical activity measurement data may include measures of physical activity, for example such as: distance moved, steps taken, and/or calories burnt. Respiratory activity measurement data may include breathing rate per minute or lung volume change measurement, for example.

The client computing device 170 may be an end user computing device, such as a laptop or a tablet or a PC or a smart phone, for example. The client computing device 170 comprises a processor 172 and a memory 174. The client device application 176 is implemented executable application program code stored within memory 174 that allows an end user to interact with the decision support software 121. The client device application 176 may include dedicated local application software (known as an “app”) or a browser application executing on the client computing device 170. The client computing device 170 also comprises a display 182 that enables the display of information to an end user and, in some embodiments, the entry of responses by the end user.

The system server 110 implements the server side part of the data fidelity management system 100 through various software modules implemented in the memory 120. The server system 110 comprises a processor 140 and a network interface 142. Processor 140 may comprise a single computer processor or multiple computer processors, and each processor may physical or virtualized or part physical and part virtual. For convenient reference herein, references to processor 140 herein should be understood to include the aforementioned variations.

The various modules implemented in the server system 110 include, for example: a clustering module 122 that performs the function of clustering information; a recommendation module 124 that performs the function of generating recommendations based on information; a fidelity management module 126 that performs the function of managing or improving the fidelity of information, for example information held in the database 190; vector representation module 128 that performs the function of representing information in vector form; a sensor device data integration module 132 that specifically handles information generated by sensors held by end-users or associated devices, such as client sensor device 160 or client computing device 170; and an NLP module 138 that performs the function of natural language processing to derive information from natural language data. The recommendation module 124 may comprise one or more models or branches for generating recommendations, for example to identify sleep problems based on input. Some of the models or branches may rely on the information in database 190 with improved fidelity to generate the recommendations or to identify sleep problems.

The network interface 142 may comprise software or hardware or a combination of software and hardware enabling the communication of the server system 110 with multiple client computing devices 170 and multiple client sensor devices 160. A database 190 may also form part of the data fidelity management system 100 and may perform the function of acting as a repository for information gathered by the server system 110 and also metrics and inferences generated by the server system 110. The database 190 may comprise a conventional relational database or a NOSQL database implemented on a separate server system. In some embodiments the database 190 may be implemented within server system 110. The server system 110 in some embodiments may be implemented on one or more virtual servers available as part of the cloud implementation. Likewise database 190 in some embodiments may be implemented as part of a cloud service implementation.

Database 190 comprises sleep disorder user data for a population of individuals. The sleep disorder user data may comprise data collected from sensors regarding the sleep patterns or other health related data capable of enabling inferences regarding sleep quality. The sleep disorder user data may also comprise information specific sleep disorders and efficacy of recommendations and treatments for individuals. The sleep disorder user data may provide a population of user data suitable for clustering, whereby sleep disorder user data of individuals may be allocated to one or more predetermined clusters depending on one or more sleep disorder, sleep pattern or sleep behaviour-based attributes. Each cluster may represent a set of individuals that share some common sleep disorder related attributes or data. The sleep disorder user data of database 190 may serve as a data set for clustering any new sleep disorder user data not previously stored in the database 190. As the sample of sleep disorder user data held in the database 190 grows over time, the predetermined clusters may be revised to take into account new sleep disorder user data received in the database 190. The revision of the predetermined clusters may comprise: identification of new clusters, merging of existing clusters, and/or reallocation of existing sleep disorder user data to a different cluster, for example.

A network 150 may include, for example, at least a portion of one or more networks having one or more nodes that transmit, receive, forward, generate, buffer, store, route, switch, process, or a combination thereof, etc. one or more messages, packets, signals, some combination thereof, or so forth. The network 150 may include, for example, one or more of: a wireless network, a wired network, an internet, an intranet, a public network, a packet-switched network, a circuit-switched network, an ad hoc network, an infrastructure network, a public-switched telephone network (PSTN), a cable network, a cellular network, a satellite network, a fiber optic network, some combination thereof, or so forth.

As of 2017, there were an estimated 92 million adults in the United States alone who have problematic sleep, equating to approximately 38% of the adult population.

One of the most prevalent of sleep problems in the U.S. is Obstructive Sleep Apnea (OSA), followed by Insomnia; however, individuals frequently experience multiple comorbid sleep problems, often choosing only to treat their most prevalent sleep problem without understanding (or acknowledging) the impact of additional sleep conditions.

In order to assist the identification of individuals' sleep problems, sleep physician consults are currently recommended. However, consults have proven to be time consuming and expensive for consumers, who may be skeptical that their unexplored sleep issues require physician intervention.

Sleep physician consults may typically involve a patient interview, in conjunction with the use of clinically validated questionnaires, including but not limited to the following: ISI: Insomnia Severity Index, Insomnia screening questionnaire, Pittsburg Sleep Quality Index, Shift Work Disorder Questionnaire, Berlin Questionnaire, MAPQ Questionnaire, Karolinska Sleepiness Scale Questionnaire, Stamford Sleepiness Scale, FSS: Fatigue Severity Scale, ESS: Epworth Sleepiness Scale, Stop Bang: an OSA assessment questionnaire, and OSA50: an OSA assessment questionnaire.

Physician consults may also include additional sleep screening elements, including but not limited to sleep diaries, single-night PSG (polysomnography), and multi-night PSG. While individual questionnaires provide for an isolated means of assessing individual sleep issues, they may not be comprehensive or sufficiently accurate for supporting decision making regarding sleep problems.

In order for individuals to attempt to carry out self-identification of their sleep disorders, they must first acclimate themselves to the range of potential sleep disorders, and then follow through by investigating the relevance of each. This poses a clear barrier to entry when determining the root cause of sleep problems. Individuals may lack awareness with respect to the range of known, common sleep issues and may not be in the best position to self-identify sleep problems independently.

When discussing their poor sleep, individuals may generally describe Insomnia or snoring type symptoms, and daytime effects, not understanding that their problem could be much more specific (OSA, delayed body clock, shift work disorder, etc.). Individuals may seek treatment recommendations from their friends or family, or search online for treatments. Very few individuals may seek advice from their PCP (Primary Care Physician), and an even smaller percentage seek out a sleep physician unless they are experiencing extreme sleep related issues.

Several PCPs acknowledge that they are not trained appropriately to help their patients with sleep related issues, other than sending patients to conduct a sleep study. During the self-identification process, individuals may choose insomnia and/or snoring products, which they often find ineffective if there has been incorrect identification of their sleep issue. They continue to follow a cycle of poor sleep and poorly chosen or ineffective solutions. Additionally, there may be a trade-off of the accuracy and reliability of self-identified issues when compared to those produced by Sleep Specialists or Physicians.

The identification of sleep disorders is often based on responses provided by individuals. The embodiments as described in the U.S. Provisional Patent Application Ser. No. 62/812,066 include examples of decision support systems that rely on response objects provided by individuals in response to query objects. The response objects may comprise information in relation to the individual's sleep disorder. There may be a subjective element in the individual's understanding of the query and the individual's formulation of a response to the query. Accordingly, the response provided may not be sufficiently reliable for a decision support system to perform its decision support operations. The subject element or the uncertainty in the responses may be described as infidelity or data infidelity.

Infidelity in subjective answers to a questionnaire may be common due to user biases and misperceptions, which are particularly prevalent in sleep problem and/or older patients. Moreover, sleep disorders, by lowering sleep quality, are generally associated with cognitive deterioration that impairs the ability of the patient to understand a query and therefore the impacts the fidelity of the answers or responses.

This may be overcome to a certain extent by asking redundant, similar or overlapping questions to confirm/validate the answers previously provided by the user, this can result in improvement in the fidelity of responses. However, there may be limits to building in redundancy into self-administered questionnaires. Redundant questions may waste the time and energy of a responder with obviously repeated questions. The described embodiments address this by determining the infidelity of responses and asking one or more redundant, similar or overlapping questions only when a certain degree of infidelity is observed. The redundant, similar or overlapping questions will generally relate to a common sleep related metric to the queries or questions originally presented to the user and that resulted in determined infidelity above a certain level. In some embodiments, the redundant, similar or overlapping questions may inform more than one sleep related metric each. However, at least one of the multiple metrics would be common to all of the redundant, similar or overlapping questions.

The similar queries or questions also should be phrased in a way so that the intent is understood by the user and the user can provide an accurate or high fidelity response. If the intent of the redundant question is unclear, the responder could easily become confused, leading to a decrease in answer fidelity. The rewordings and the order in which they are added to the survey must also be carefully examined to prevent the introduction of biases (such as order bias or halo effect bias) into the queries.

The described embodiments identify redundant questions in the response objects comprising the responses provided by a user or individual, calculate the level of answer infidelity, and when a certain threshold of answer infidelity is reached, present additional query objects to clarify the response in an effort to reduce sleep disorder data infidelity.

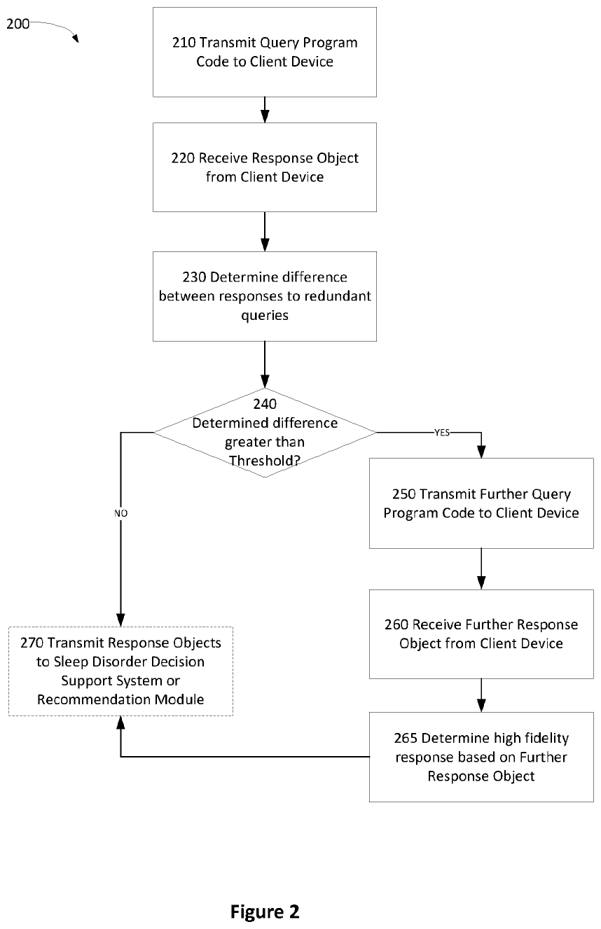

FIG. 2 is a flowchart of a method 200 of improving the fidelity of sleep disorder data. At step 210, query program code is transmitted to a client device 170 by the server system 110 over the network 150. The query program code when executed by the client device 170, make accessible to a user on more queries embedded in the query program code. The user may interact with the queries by provide one or more responses that are transmitted by the client device 170 and received by the server system 110 at step 220.

At step 230, the server system 110, through the fidelity management module 126 determines or quantifies any infidelity metric or inconsistency metric between the received responses. At step 240, the fidelity management module 126 asses if the determined infidelity metric exceeds a threshold. If the determined infidelity metric exceeds a threshold, then further query program code is transmitted to the client device at step 250. The further query program code comprises query objects that clarify or invoke a more clear response that is received by the sever system 110 at step 260 as a further response object. At step 265, the further response object is assessed and is used to substitute the low fidelity responses previously obtained. The steps 250, 260 and 265 may be repeatedly performed if more than one instances of higher than threshold infidelity are determines at steps 230 and 240.

The validated lower infidelity responses obtained at step 265 are transmitted to a sleep disorder decision support system for further analysis. In some embodiments, the validated lower infidelity responses obtained at step 265 may be transmitted to the recommendation module 124 that serves as part of a sleep disorder decision support system.

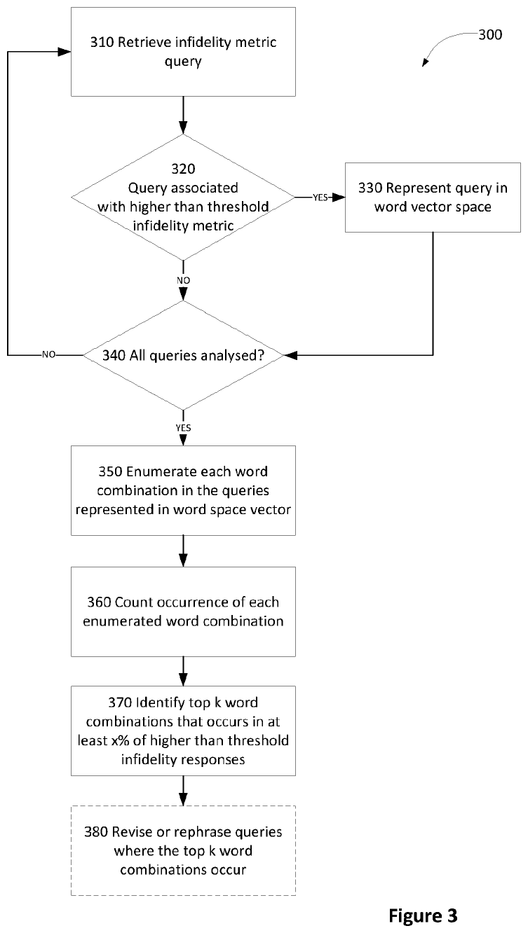

FIG. 3 is a flowchart of a method 300 for revising queries to improve fidelity of sleep disorders data. This method is performed by analyzing responses to query objects obtained over time. At step 310, the fidelity management module 126 analyses responses to a set of query objects over time and determines an infidelity metric for each query. The infidelity metric represents the degree of inconsistency of responses to the query over time. Accordingly, queries with higher infidelity metrics are considered deserving of revision to reduce the infidelity of futures responses and improve the conciseness and reliability of the entire query set.

At step 320 if a query is associated with a higher then threshold infidelity metric, then control passes on to step 330. At step 330 the query is represented in the form of a word space vector, which can also be described as word embedding. The word space vector may comprise a mapping of words in a query to a set of most common dictionary words. Accordingly, the representation of a query in the form of a word space vector identifying the occurrence or non-occurrence of a set of common words in the query.

At step 340, the Fidelity Management Module 126 determines if all the queries that are part of the data fidelity management software 121 have been analysed. If further queries remain to be analysed, then execution continues at step 310. Otherwise at step 350, after all the high infidelity queries transformed into word space vectors, each word combination in the word space vectors is separately enumerated. This enumeration step identifies all possible words or word combinations that may contribute to high infidelity of responses across all the queries.

At step 360, the occurrence of each enumerated word combination across all the queries is counted. At step 370, the top k word combinations that are related to a infidelity metric of greater than x % are separately identified as causative of the higher infidelity. The further optional step of 380 may comprise a revision or rephrasing of the queries that comprise the identified word combinations causative of higher infidelity.

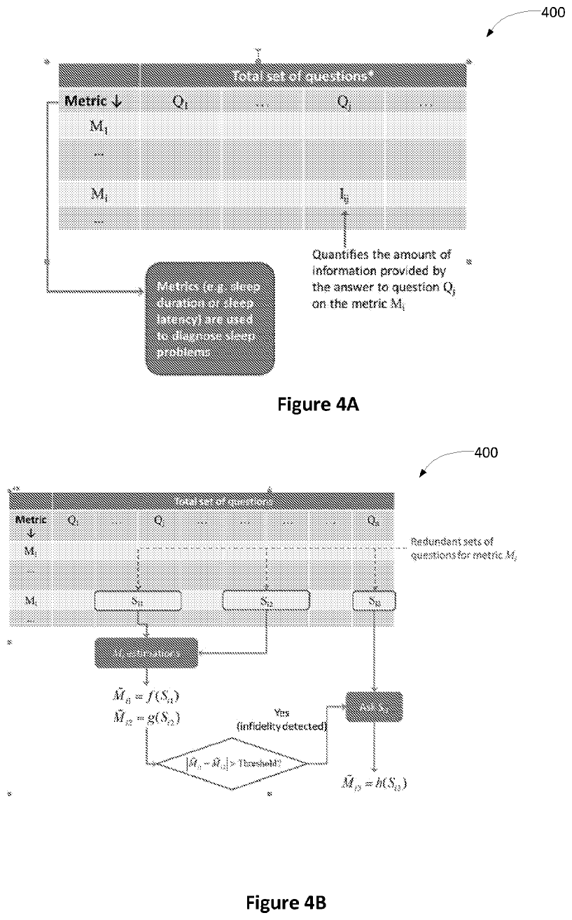

FIG. 4 is a matrix 400 showing part of the operation of method 200. Matrix 400 comprises a mapping between relevant “Mi” (sleep metric, e.g. sleep duration, sleep latency, sleep state prior to waking-up, or sleep-stage durations) and questions or queries “Qj”. Sleep metrics may comprise various measures for quantifying aspects of the sleep of an individual. Sleep metrics may include: time an individual went to bed, time an individual got out of bed, sleep onset time, wake up time, time an individual spent in each sleep stage, for example. Each time value or metric may be represented as a time data record including data associated with a particular time zone in a 24 hour time format, for example. A measure of the amount of information provided by the answer to question “Qj” in metric “Mi” is referred to as “Iij”. The measure of the amount of information values “Iij” may indicate the significance of the answer to question “Qj” in quantifying or assessing metric “Mi” for an individual. In some embodiments, some of the queries Qi may be directed to the sensor device 160 or the sensor 180.

For example, a question directly asking about average sleep duration informs the sleep duration metric, and the combination of answers to questions “when do you go to sleep” and “when do you wake-up”, does also inform the sleep duration metric. The first question constitutes a first query subset, and the last two questions constitute a second query subset informing the sleep duration metric. A third query subset may be directed to the result(s) of sensor data to measure sleep duration. In this example, the third query subset may provide the greatest amount of information on sleep duration (without loss of generality). For this example, the Iij value for the third query subset may be set to 1. These three query subsets may be considered to be redundant because they inform a single common metric.

FIG. 4B illustrates an example of the application of the method of FIG. 2 using the matrix 400. For example, a given sleep related metric “Mi” may be informed by three query subsets: Si1, Si2, and Si3 which are redundant and (directly or indirectly) inform the same metric. Considering a sleep disorder for which “Mi” is relevant and the answers to questions in the Si1, and Si2 subsets lead to substantially different estimations of Mi (which reflect infidelity), then the questions in subset Si3 may need to be presented to reduce infidelity. The presentation of the further query subset Si3 may occur at step 250 of FIG. 2. If, in some embodiments, only two subsets Si1, and Si2 are available that relate to metric Mi, another possible course of action if discrepant “Mi” estimations are found, comprises asking users to verify their answers. In other words, one or more of the additional queries to reduce infidelity may also be identical or generally similar to one of the previous queries presented to a user where the presentation of such a query is determined by software 121 to likely to result in a higher fidelity response (i.e. lower difference score) relative to the previous responses.

As an example, for sleep maintenance insomnia disorders, it is relevant to know the number of times users wake-up within a sleep session. Therefore, it is possible to directly ask patients to report the average number of times they wake-up within a typical sleep session, and also submit sensor obtained data including the clock times at which they wake-up. The latter can be used to verify fidelity; e.g. if the patient reports waking-up three times and the objective data reports a single time, the patient may be asked to check her answer to ensure higher accuracy.

In some embodiments, the user's answers to the queries in an initial questionnaire are designated as responses to “Questionnaire A”. As the answers are processed by an algorithm, the user's responses may be compared with pre-collected sensor based data or additional redundant questions, to calculate a difference between initial responses and re-collected sensor based data or additional redundant questions. Once this delta (ie. the difference) hits a pre-determined threshold, (“Threshold X”) the software 121 sends additional query sets to the client device 170, in order to reduce the level of answer infidelity to an acceptable lower level (“Threshold Y”, which is lower than Threshold X).

Additionally, the amount or measure of answer infidelity can be tracked by the fidelity management module 126 and later analyzed as part of an optimisation procedure to identify particular sets of redundant questions that consistently produce answer infidelity above Threshold X. Later, an identified set of redundant questions may be used to identify questions with ambiguous wording or unclear intent. Difficult-to-understand questions may lead to infidelity for a substantial portion of users (e.g. >20%) in the system.

Answer infidelity may be quantified or measured by determining an average difference between answers to redundant, similar or overlapping queries that relate to a common metric. The average difference may be determined over a subset of the population of users or a specific cluster of the population of users or over the entire population of users. The cluster of users may be based on demographics, for example. The determined average difference may be used as a benchmark or basis to define a threshold. The threshold may be used to assess the fidelity of a specific response to a query object.

For example, for a population of users, the average difference between responses to two redundant query objects relating to a total sleep time metric may be 1.5 hours. Accordingly, a threshold may be defined based on the average difference of 1.5 hours. The threshold may be for example equal to the average difference (1.5 hours), or may be defined by a ratio or a multiplier (for example a selected predetermined ratio of 1.2 applied to the determined average may mean the threshold is set at 1.8 hours). The ratio may be determined based on experimentation with sleep disorder data from a representative population. If for a particular user, the difference between responses to two redundant, similar or overlapping query objects relating to a total sleep time metric is 2 hours, assuming that the threshold is either 1.5 hours or 1.8 hours, then infidelity may be flagged or indicated or determined with respect to the response of the particular user because the difference in the user's response (2 hours) is greater than the defined threshold (1.5 hours or 1.8 hours for example). Likewise, infidelity may not be flagged with respect to the responses of a particular user if the difference between the responses to two redundant query objects if less than the defined threshold for the same two query objects for a population of users.

The identified questions can then be (automatically or manually) updated to improve clarity and thus decrease the amount of answer infidelity. Over time, rewording questions or providing additional information to clarify them may lead to a decrease in the number of confusing questions and, ultimately, convergence of a list of optimally worded questions with clearer meaning.

More accurate information from the users may ultimately result in an improved accuracy of the sleep disorder decision support system or the recommendation module 124, and in turn, increase the success rate with any recommendations made to improve a user's sleep problem(s).

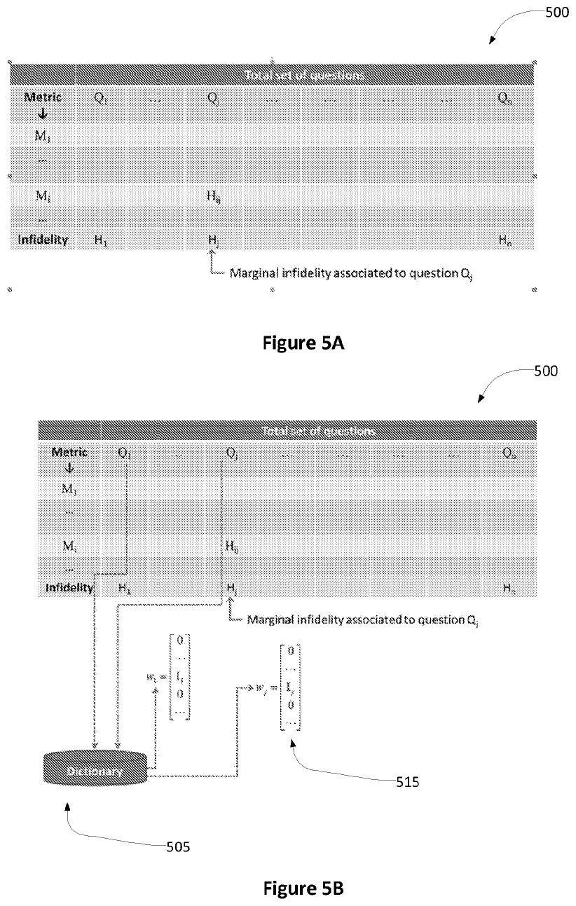

In some embodiments, the fidelity management module 126 may be configured to build a map of infidelity where the average infidelity for each metric-question combination is estimated across a population of users. Matrix 500 in FIG. 5A shows an example of a map of infidelity in the form of a matrix.

By integrating the average infidelity metric across columns (i.e. the question dimension), a marginal infidelity (Hi) associated with each question can be estimated.

While FIG. 5A illustrates the analysis of infidelity at the question level, it is also possible to analyze the questions at the word level to identify word combinations that lead to infidelity in responses as described with reference to the flowchart of FIG. 3. This can be accomplished by projecting questions into a word-space vector defined by a dictionary containing “V” most common words. Such projection may lead to sparse vectors with V components with entries equal to 1 only at positions corresponding to words present in a given question. This information is useful in designing future questions, and in assigning extra attention toward questions containing infidelity-inducing word combinations.

FIG. 5B illustrates parts of the steps of method 300 of the flowchart of FIG. 3. FIG. 5B also illustrates a dictionary 505 which may be a list of most common words that form the basis for projecting a question or query into a word space vector. An example of the projection of question Qj is the vector wj 515.

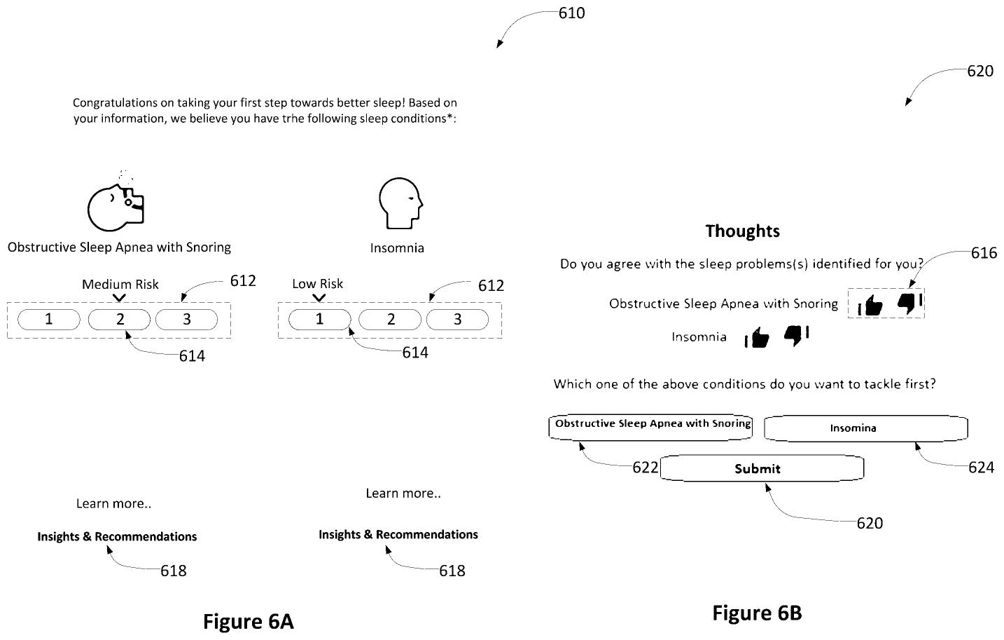

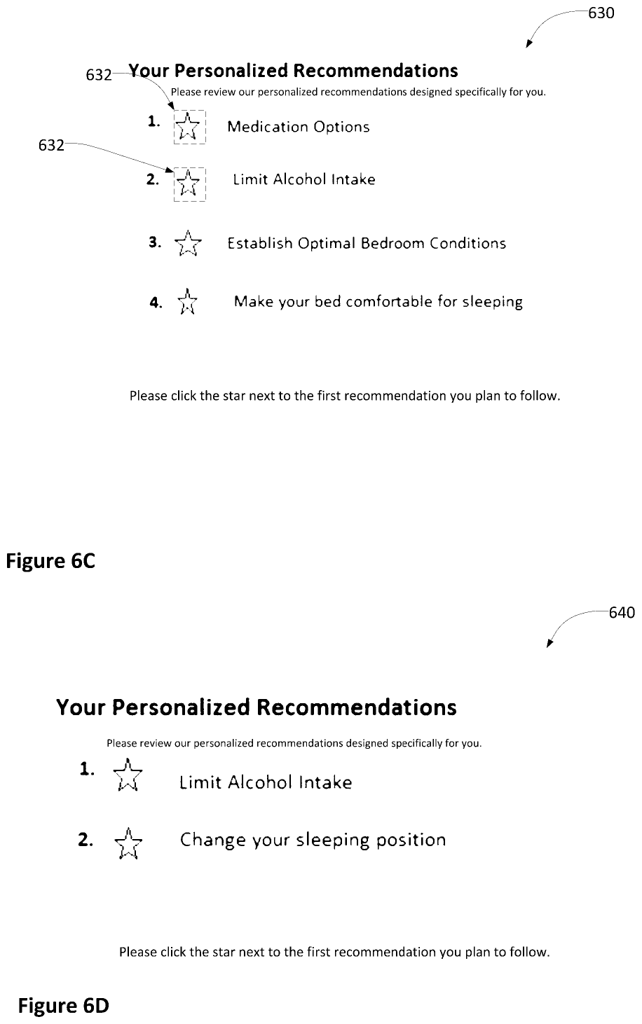

FIGS. 6A, 6B, 6C and 6D are example screenshots represented on the display 182 of the client computing device 170. FIG. 6A is an example screenshot display 610 showing a representation of sleep problems identified in relation to an end user. Region 612 of display 610 represents the graded scale of risk level the respect to a particular sleep disorder. A risk level indicator 614 in region 612 indicates the level of risk of an end user with respect to an identified sleep disorder. The different displayed levels of risk may be low, medium or high, for example. The display 610 may also comprise a selectable icon 618 allowing the display of further insights and recommendations in relation to a particular sleep disorder identified in display 610.

The region 612 may be served by the client device 160 in response to program code received by the client device 160 from the server system 110. Each of the risk level indicator 614 may be a dynamic programmable regions whose appearance or illumination may vary dynamically depending on the program code received by the client device 160.

Screenshot 620 of FIG. 6B represents an example interface display 620 at client computing device 170 allowing an end user to provide feedback input in relation to one or more identified sleep disorders on display 620. In some embodiments, the feedback input may be provided by clicking on thumbs up or thumbs down icons 616 in relation to each identified sleep disorder. Once the user has specified their feedback input at client computing device 170, for example using the selectable thumbs up or thumbs down icons 616, the user may click on the submit button 620 to submit the feedback input.

Screenshot 620 also comprises dynamic regions 622 and 624 that may correspond with a sleep disorder. The dynamic selectable regions 622 and 624 may be rendered by the client device 160 depending of the program code received by the client device. For example, in the screenshot 620, region 622 corresponds to Obstructive Sleep Apnoea with Snoring and region 624 corresponds to insomnia. The regions 622 and 624 may dynamically vary depending on the program code received by the client device 160 in relation to the respective regions.

Screenshots 630, 640 in FIGS. 6C and 6D respectively illustrate an interface displaying recommendations in relation to identified sleep disorders for an end user. The interface as displayed in the screenshots 630 and 640 also includes selectable marker icons 632. The selectable marker icons 632 may allow an end user to subscribe to particular recommendation or provide feedback to the decision support system 100 in relation to a particular recommendation. Selection of such marker icons via the client computing device 170 is recorded by recommendation module 124.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A computer-implemented method for fidelity improvement of sleep disorder data, the
method comprising:
a server system transmitting query program code defining a plurality of queries to a client device, the plurality of queries relating to sleep disorders, wherein:
at least two queries of the plurality of queries relate to a sleep metric that is common to the at least two queries, and at least one other of the plurality of queries relates to a different sleep metric, and
the query program code is executable by the client device to permit the client device to transmit at least one response object encoding a response to each of the at least two queries;
the server system receiving the at least one response object from the client device and determining the response to each of the at least two queries encoded in the response object;
determining a difference between the responses to the at least two queries that relate to the common sleep metric;
responsive to determining that the difference is greater than a pre-determined difference threshold, the server system transmitting further query program code defining a further query that relates to the common sleep metric, the further query program code being executable by the client device to permit or cause the client device to transmit a further response object encoding a further response to the further query, wherein the further query is selected so that a difference between the further response and one of the at least two queries is less than the predetermined difference threshold;
the server system receiving the further response object from the client device and determining the further response encoded in the further response object.

2. The method of claim 1, further comprising determining the difference threshold,
wherein the difference threshold is determined based on response values of a plurality of stored response objects related to the common sleep metric accessible to the server system.

3. The method of claim 2, wherein the common sleep metric and the different sleep
metric are part of a plurality of sleep metrics, wherein each sleep metric has a respective difference threshold associated therewith.

4. The method of claim 1, wherein the further query program code is
directed to a sensor and the further response object comprises health data from the sensor.

5. The method of claim 4, wherein the client device comprises the sensor.

6. The method of claim 4, wherein the sensor is separate from the client device and the
client device communicates with the sensor to receive data content of the further response object.

7. The method of claim 4, wherein the health data comprises one or
more of: cardiac activity measurement data, physical activity measurement data, blood pressure measurement data, respiratory activity measurement data, brain activity data, eye movement data, sleep stage data, heart rate data, movement data, breathing sound data, breathing rate data or other longitudinal biological measures.

8. A computer-implemented method for revising queries to improve fidelity of sleep
disorder data, the method comprising:
retrieving infidelity metric data in relation to a plurality of queries relating to sleep disorders and in relation to responses provided to the plurality of queries;
for each query in the plurality of queries that has an infidelity metric value greater than a predetermined infidelity threshold, generating a word space vector of the query;
enumerating all possible word combinations in each of the word space vectors;
counting occurrence of each enumerated word combination across all of the word space vectors;
identifying a subset of the enumerated word combinations having an occurrence greater than an occurrence threshold;
revising each query in the plurality of queries where the identified subset of the enumerated word combinations occur to improve the fidelity of responses to each revised query.

9. The method of claim 8, wherein the generated word space vector is a sparse vector.

10. The method of claim 8, further comprising:
a clustering module of the server system identifying one or more clusters of sleep disorder user data that most closely relate to the retrieved infidelity metric data;
wherein the revising comprises, for each query in the plurality of queries, revising that query for each of the identified one or more clusters.

11. The method of claim 8, wherein the infidelity metric data
comprises health data from a sensor.

12. The method of claim 11, wherein the health data comprises one or more of:
cardiac
activity measurement data, physical activity measurement data, blood pressure measurement data, respiratory activity measurement data, brain activity data, eye movement data, sleep stage data, heart rate data, movement data, breathing sound data, breathing rate data or other longitudinal biological measures.

13. A system for fidelity improvement of sleep disorder data, the system comprising a
server system comprising:
one or more processors;
a memory accessible to the one or more processors storing executable program instructions to implement the method of claim 1.

14. A system for revising queries to improve fidelity of sleep disorder data, the system
comprising a server system comprising:
one or more processors;
a memory accessible to the one or more processors storing executable program instructions to implement the method of claim 8.

15. One or more computer-readable media storing computer-executable instructions that,
when executed by one or more computers, direct the one or more computers to perform the method of claim 1.
